(19) United States
(12) Patent Application Publication
(10) Pub. No.: US 2010/0099952 A1
(43) Pub. Date: Apr. 22, 2010

(54) Steerable Shaft Having Micromachined Tube

(75) Inventor: Mark L. Adams, Sandy, UT (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: BOSTON SCIENTIFIC SCIMED, INC., Maple Grove, MN (US)

(21) Appl. No.: 12/582,288

(22) Filed: Oct. 20, 2009

Related U.S. Application Data
(60) Provisional application No. 61/107,444, filed on Oct. 22, 2008.

(51) Int. Cl. A61B 1/005; A61M 25/092; B23P 17/04

(52) U.S. Cl. 600/146; 604/95.04; 29/428

(57) ABSTRACT

A shaft of a steerable medical device includes a torque tube that allows the tip of the shaft to be torqued from the proximal end. In one embodiment, the torque tube includes a metal tube having a series of opposing cuts therein to form a number of axially aligned rings that are joined by spacing beams. The cuts are oriented in different directions along the length of the torque tube to allow bending in any direction and effective transfer of rotational torque.

### CROSS-REFERENCE TO CO-PENDING APPLICATIONS

This application claims the benefit of priority under 35 U.S.C. §119(e) of U.S. Provisional Application No. 61/107,444, filed Oct. 22, 2008, the entire disclosure of which is incorporated herein by reference.

### BACKGROUND

As an alternative to performing more invasive types of procedures in order to examine, diagnose, and treat internal body tissues, many physicians are using minimally invasive devices such as catheters and endoscopes to perform such tasks. Such medical devices have shafts that are partially inserted into the body and routed to a point of interest in order to allow the physician to view and treat internal body tissues. Generally, such shafts include two or more control cables to steer the tip of the device through passageways of the human anatomy.

When four cables are used, one pair of cables steers the tip of the shaft in one plane and a second pair of cables steers the tip in a second plane and perpendicular to the first plane, thereby providing the ability to steer the tip in four directions. A four-way steerable shaft is advantageous when negotiating a tortuous passageway of human anatomy. A four-way steerable shaft is also very flexible to facilitate steering and reduce tension on the control cables. However, because of the highly flexible construction, the shaft may deform under low torque (rotational) forces. Furthermore, because of the multitude of control cables, the diameter of the shaft that is needed to accommodate four control cables, a working channel, and other lumens that supply air and liquids, may preclude the medical device from being used in the narrow passageways of human anatomy.

### SUMMARY

A steerable medical device is provided with an elongated shaft having the ability to be torqued (i.e., rotated) at the proximal end such that the torque is transferred along the shaft to the distal end. The ability of the medical device to be torqued or rotated, wherein torque forces are transferred the length of the shaft from the proximal section to the distal end of the shaft, allows the device to be fabricated with fewer control cables. A single control cable or a pair of control cables can be used to steer the shaft tip in one plane, while the ability to torque the device can be used to orient the tip in a multitude of other planes, thus providing the functionality of four-way steerable devices but with fewer control cables. With the omission of control cables, the outside diameter of the shaft can be reduced, therefore increasing its ability to be inserted and tracked through small passageways of the human anatomy. Alternatively, the medical device can be fabricated having the same outer diameter as a medical device having two pairs of control cables. However, the greater cross-sectional area gained can be used to provide additional or larger working channels or more functionality by including greater numbers of lumens in the shaft.

In one embodiment, the shaft of the medical device is constructed having a hollow, flexible torque tube that extends from the proximal section to the distal section. The torque tube includes sets of a number of rings connected together by axial beams along the length of the tube that results in the tube being flexible yet still allows the transfer of rotational forces from the proximal section. The torque tube is torsionally rigid so that no significant deflection occurs circumferentially under a normal medical procedure.

In one embodiment, the axial beams and rings are a result of making a series of cuts from opposite directions along the length of the torque tube. Opposed cutting elements or saw blades may be used in making the cuts that are aligned perpendicular to the longitudinal axis of the tube.

This summary is provided to introduce a selection of concepts in a simplified form that are further described below in the Detailed Description. This summary is not intended to identify key features of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagrammatical illustration of a conventional steerable medical device with four control cables;

FIG. 2 is a diagrammatical illustration of a steerable medical device having a torque tube in accordance with one embodiment of the present invention;

FIG. 3 is a diagrammatical cross-sectional illustration of a shaft without a torque tube;

FIG. 4 is a diagrammatical cross-sectional illustration of a shaft with a torque tube in accordance with one embodiment of the present invention;

FIG. 5 is a diagrammatical cross-sectional illustration of another embodiment of a shaft without a torque tube;

FIG. 6 is a diagrammatical cross-sectional illustration of a shaft with a torque tube in accordance with an embodiment of the present invention; and

FIG. 7 is a diagrammatical illustration of a torque tube in accordance with one embodiment of the present invention.

### DETAILED DESCRIPTION

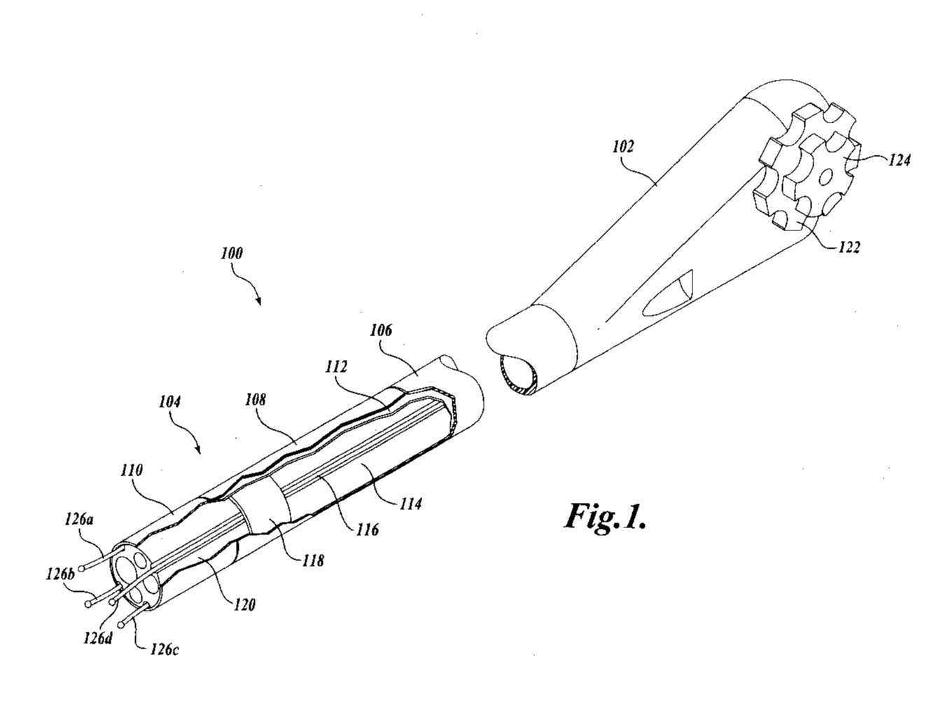

FIG. 1 is an illustration of a steerable medical device 100. The medical device 100 includes a handle 102 and a flexible shaft 104. The shaft 104 includes a proximal section and a distal section. The proximal section of the shaft 104 is connected to a distal end of the handle 102. The tip at the distal section of the shaft 104 is steerable by means of a number of control cables 126a-d on the inside of the shaft 104. Control cable 126a is paired with 126c; control cable 126b is paired with 126d. One pair of cables controls the direction of the tip of the shaft 104 in a single plane. With four cables, i.e., two pairs of cables, the tip of the endoscope shaft 104 can be steered in any combination of four directions (four degrees of freedom). In the embodiment shown, the first and second pairs of control cables are controlled by steering dials 122 and 124 on the handle 102. One particular embodiment of a shaft 104 includes an outer cover or sheath 106. The sheath 106 can be made from an elastomer, such as a polyether block amide (PEBA) or other suitable material. A representative polyether block amide is known under the designation PEBAX®. PEBAX® is an elastomer whose characteristics are determined by a number that follows the PEBAX® name. PEBAX® 7233 is suitable for the sheath 106 at the proximal section of the shaft 104. PEBAX® 3533 is suitable for use at about the central section of the shaft 104. PEBAX® 7233 may be used again at the distal section of the shaft 104. A metal braid mesh 112 is adjacent to or incorporated into the sheath 106.

Within the sheath is a multi-lumen tube 114. In one particular embodiment, the multi-lumen tube 114 includes eight lumens. Lumens can be used for delivery of air or vacuum, fluids, liquids, and external devices to the distal tip of the shaft 104. In a conventional four-way steerable shaft, four of the lumens are reserved for control cables 126a, 126b, 126d, and 126c. The multi-lumen tube 114 can be extruded from an elastomer, such as a polyether block amide. The components illustrated in FIG. 1 either alone or in combination do not provide the shaft 104 with sufficient strength to enable the transfer of rotation to the distal end of the shaft 104.

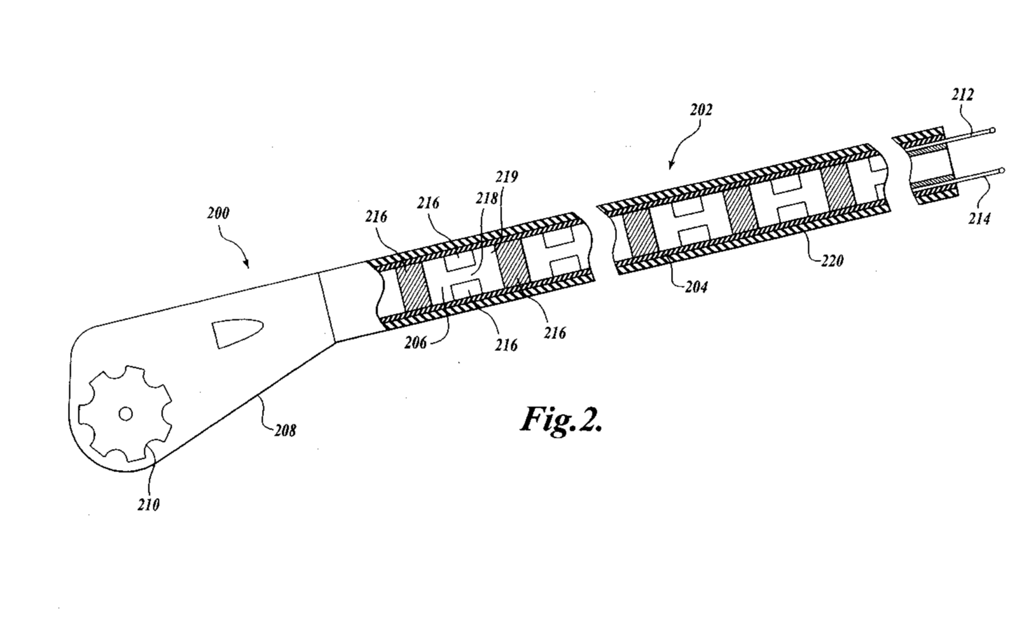

Referring to FIG. 2, a steerable medical device 200 made in accordance with one embodiment of the present invention is illustrated. In addition to the shaft components illustrated in FIG. 1, the device shown in FIG. 2 additionally includes a torque tube 206. The medical device 200 includes an elongate shaft that may be coupled at its proximal end to a handle 208. A braid mesh 204 may cover the torque tube 206. An outer sheath 220 may cover the braid mesh 204. A multi-lumen tube (not shown) may be positioned within the torque tube 206. While the medical device 200 is illustrated having various components in a particular configuration, the medical device may have fewer or additional components that can be arranged in any sequence. The endoscope 200 is merely representative of one embodiment. Other suitable medical devices may be found in U.S. patent application Ser. No. 10/604,504 filed Jul. 25, 2003, now U.S. Pat. Publ. No. US 200410181174 A2, which claims the benefit of priority to U.S. Provisional Application No. 60/399,046, filed Jul. 25, 2002; U.S. patent application Ser. No. 10/914,411 filed Aug. 9, 2004, now U.S. Pat. Publ. No. 2006/0030753 A1; U.S. patent application Ser. No. 11/388,247 filed Mar. 23, 2006, now U.S. Pat. Publ. No. 2006/0252993 A1; and U.S. patent application Ser. No. 11/089,520 filed Mar. 23, 2005, now U.S. Pat. Publ. No. 2005/0272975 A1, the entire disclosures of which are all hereby incorporated by reference.

Torque tube 206 is made from a rigid or semi-rigid material to permit transferring rotational torque forces from the proximal end to the distal end of the shaft 202 without significant deflection circumferentially. Materials from which torque tube 206 is made include, but are not limited to, metals, including nickel, titanium, stainless steels, etc., and their alloys; polymers, such as poly(acrylonitrile butadiene styrene), polycarbonate, and high density polyethylene. In one embodiment, a nickel-titanium compound is a shape memory metal known as Nitinol. Medical device 200 may have a single pair of control cables 212 and 214 generally located directly opposite to each other, which cables may be coupled to a single steering dial 210. Steering dial 210 functions with cables 212 and 214 to steer the tip of the shaft 202 in a desired direction. Torque tube 206 functions to rotate the tip of the shaft 202 in a direction from rotational movement imparted toward the desired proximal end of the shaft such as at the handle 200. Therefore, to steer the tip of the shaft 202 in a direction that is not in the plane steerable by the control cables 212 and 214, the shaft 202 can be rotated from a proximal location. In this manner, the device 200 functions as a four-way steerable endoscope or guide tube with a single pair of control cables.

In an alternative embodiment of the invention, the medical device 200 can have a single control cable to steer the tip and a spring can bias the shaft tip in the opposite direction. Such a spring may be integral to the torque tube. For example, the torque tube may be naturally resilient such that it springs back to its straight or undeflected shape when free from external forces such as those that may be imparted by the control cable. Alternatively, a torque tube, as described herein, may include a resilient material in the cut and attached to the opposite laterally extending sides of the cut. Such a resilient material may act as a spring in both tension and compression and may be added to control the resiliency of the torque tube without affecting its flexibility. An elastomer or rubber material may be suitable for such a use and would not expand the outer diameter or reduce the available space inside the torque tube. Indeed, by eliminating one of the control cables, the space available inside the torque tube may, in fact, be enlarged as compared to a medical device having a torque tube of the same diameter and two control cables.

In one embodiment, the torque tube 206 may include cuts 216 made at regular or at irregular intervals along the length of the torque tube 206. The function of cuts 216 is to bias a tube 206 to have the ability to flex, while also being capable of transferring rotational torque forces from a proximal location to the distal tip of the shaft 202. Adjacent cuts 216 along the length of torque tube 206 may be rotated from each other from 0 degrees to 90 degrees so that the axes of adjacent cuts are perpendicular. In one embodiment, the cuts 216 are made in pairs from opposite sides of the torque tube 206 and leave a thin axial beam 218 between a pair of cuts and a ring 219 from adjacent cut to adjacent cut. The beam 218 may be longitudinally aligned in comparison to the long axis of the tube 206, whereas the ring 219 may be transversely aligned in comparison to the long axis of the tube 206. The thin beam 218 of material between pairs of cuts 216 allows the tube 206 to articulate, i.e., flex, at the beam 218. By changing the orientation of cuts 216 along the length of the shaft 202, the shaft 202 may be made flexible in all directions. In one embodiment, each pair of rings 219 are separated by diametrically opposed beams 218. The beams may further be aligned so that adjacent pairs of beams are oriented at 90° to each other. The torque tube 206 is designed to the ability to rotate the distal tip of the shaft 202 by torquing the proximal end of the shaft 202. Thus, a single pair of control cables 212 and 214 in combination with the torque tube 206 is designed to produce the functionality of a four-way steerable shaft with four control cables, such as shaft 104 of FIG. 1. Therefore, a pair of control cables can be eliminated.

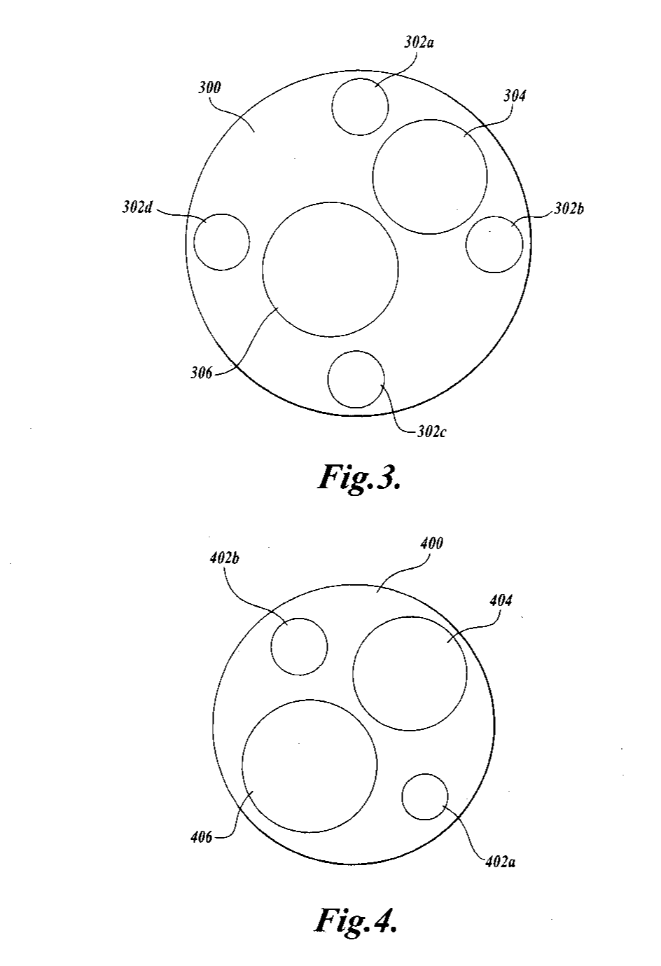

FIG. 3 is a cross-sectional illustration of a multi-lumen tube 300 that can be used for shafts having four control cables. The tube 300 can be made of an elastomer, such as an extrusion of polyether block amide. However, those skilled in the art will appreciate that other elastomers or materials may be used. The tube 300 may include six lumens. In some embodiments, four of the lumens 302a, 302b, 302c, and 302d are reserved for control cables to enable four-way steering with four control cables. Lumens 302a, 302b, 302c, and 302d may be placed at four positions: 0 degrees, 90 degrees, 180 degrees, and 270 degrees. Because of the required placement of the lumens 302a, 302b, 302c, and 302d, lumens 304 and 306 may be arranged wherever possible in the remaining cross-sectional area. Consequently, the maximum diameters of lumens 304 and 306 are limited by the presence of the four lumens for the control cables.

Referring to FIG. 4, a cross-sectional illustration of a multi-lumen tube 400 for a shaft of a steerable medical device such as an endoscope is illustrated. The multi-lumen tube 400 may be incorporated into a shaft that is constructed as shown in FIG. 2. The multi-lumen tube 400 can be made from an elastomer. The multi-lumen tube 400 of FIG. 4 is incorporated into a shaft having a torque tube, such as torque tube 206 shown in FIG. 2. The multi-lumen tube 400 can include the lumens 402a and 402b placed generally opposite to each other. Lumens 402a and 402b can accommodate a pair of control cables (not shown). A pair of control cables can steer the tip of an endoscope shaft in a plane. Alternatively, a single control cable can be used when paired with a biasing device that opposes the movement of the single control cable. While a shaft having a single control cable with a spring bias or a pair of control cables provides steering in one plane, when used with a shaft having a torque tube, the device can be selectively oriented in any of the left/right, up/down directions. The torque tube 206 of FIG. 2 can be used to apply torque at the proximal end of the shaft 202 with the handle 208. Accordingly, utilizing a torque tube 206 can advantageously result in fewer lumens without sacrificing a highly steerable or highly directional shaft tip. Furthermore, as can be seen by comparing the tube 300 of FIG. 3 with the tube 400 of FIG. 4, the tube 400 of FIG. 4 can have a smaller outer diameter by the elimination of two of the lumens necessary for control cables. Comparing the cross-sectional configuration of tube 300 in FIG. 3 with the cross-sectional configuration of tube 400 in FIG. 4, it can be appreciated that the outer diameter of the tube 400 is likely to be less than the outer diameter of the tube 300. However, the tube 400 has similar sized lumens 404 and 406. The lumens 404 and 406, including a working channel, can remain essentially the same diameter as the lumens 304 and 306 of the larger tube 300 having four steering cables. The torque tube 206 may be made from a thin walled tube that will not add significantly to the overall diameter of the shaft. Accordingly, by having a torque tube, the outside diameter of an endoscope shaft can be reduced, therefore allowing the shaft to be used in the small passageways of human anatomy, such as the bile ducts.

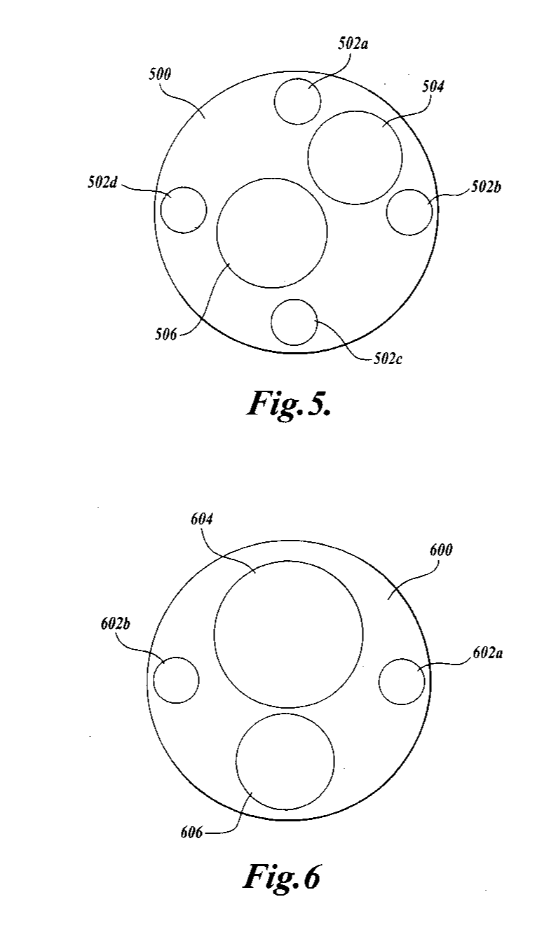

Referring to FIG. 5, a cross-sectional view of a multi-lumen tube 500 having six lumens, four of which may be dedicated to control cables, is illustrated. The lumens 502a, 502b, 502c, and 502d are for control cables. The lumens 502a, 502b, 502c, and 502d are positioned at 0 degrees, 90 degrees, 180 degrees, and 270 degrees. Lumens 504 and 506 are also provided, one of which may be a working channel. The maximum diameters of lumens 504 and 506 are limited by the presence of lumens 502a, 502b, 502c, and 502d.

Referring to FIG. 6, a cross-sectional illustration of a multi-lumen tube 600 having the same outer diameter as tube 500 is illustrated. Tube 600 may have four lumens, only two of which, lumens 602a and 602b, are dedicated to control cables. Lumens 602a and 602b are positioned at 90 degrees and 180 degrees, respectively. However, it will be appreciated that any other orientation can be used, as long as lumens 602a and 602b are substantially opposite to one another. The tube 600 has a similar outer diameter as tube 500 of FIG. 5. Tube 600 may be incorporated into a shaft that includes a torque tube, such as torque tube 206 of FIG. 2. Because of the ability to torque the shaft of the endoscope with the torque tube 206, two of the lumens in device 500 for control cables can be eliminated. With the additional cross-sectional area that is made available by the elimination of two lumens, the lumens 604 and 606 can be made larger as compared with lumens 504 and 506 of the tube 500 having the same outer diameter. Therefore, a larger working channel can be provided in the same cross-sectional area as compared with a multi-lumen tube having lumens for four control cables. Alternatively, the working channel can remain the same and additional lumens may be added for increased functionality.

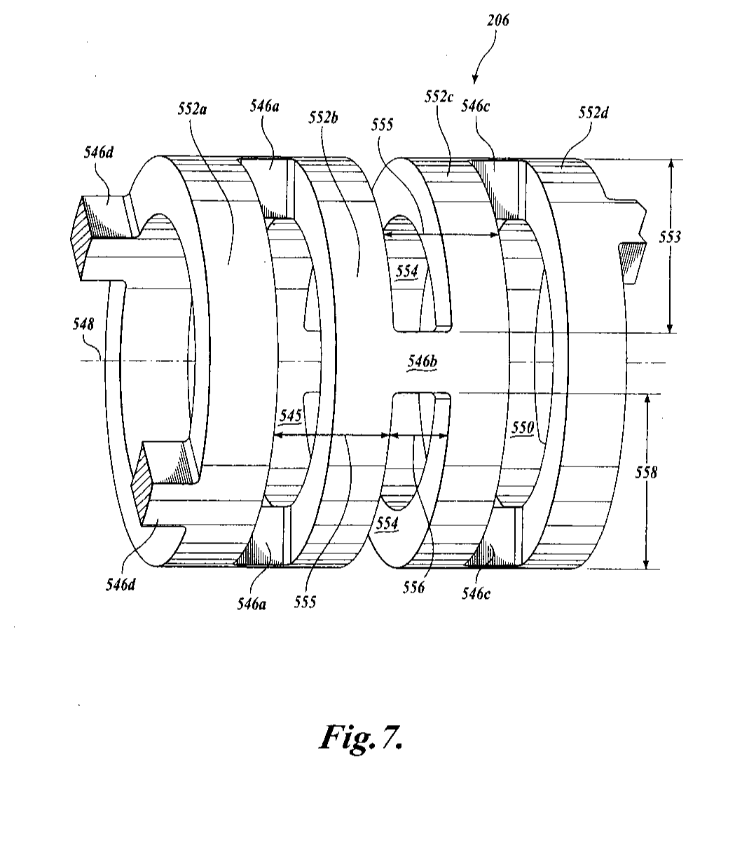

In FIG. 7, one representative embodiment of a section of a hollow and flexible torque tube 206 is illustrated. One representative method for making a torque tube is described in U.S. Pat. No. 6,766,720, to Jacobsen et al., herein expressly incorporated by reference. Jacobson and other inventors, namely Davis and Snyder, hold numerous U.S. patents and U.S. patent publications that describe “micromachining” technology and related uses, including: U.S. Pat. Nos. 5,106,455; 5,273,622; 6,428,489; 6,017,319; 6,440,088; 6,478,778; 6,014,919; 6,063,101; 6,022,369; 6,138,410; 6,302,870; 6,214,042; 2002/0082499; 2003/0009208; 2004/0111044; and 2004/0181174. All the aforementioned patents and publications are incorporated herein expressly by reference for all purposes. The method of making torque tubes described in Jacobsen et al. is referred to as a micromachining method. Torque tubes have the ability to flex (sideways motion) while also providing the ability to be torqued, such that torque forces will be transferred along the length of the tube. Incorporating a torque tube into a shaft of a medical device, such as an endoscope, has the advantage that fewer control cables are required, thus, freeing cross-sectional area for reducing the outside diameter of the shaft or, alternatively, maintaining the same diameter but providing larger diameter lumens and working channels or greater numbers of lumens. Torque tubes are highly flexible, but can transmit torsional forces along the length of the tube from the proximal section to the distal section without significant deflection circumferentially. A section of a micromachined tube 206 having cuts formed therein is illustrated in FIG. 7.

One alternative embodiment (not illustrated) is similar to any of the medical devices described above, but includes a pre-bent torque tube such that one or more of the steering cables may be used to straighten the torque tube to a non-bent condition as well as to control the tip of the device as described above.

The method for making a torque tube for use in an endoscope includes making a series of two cuts from opposite sides of the tube 206 at the same location along the longitudinal axis 548 of the tube. The depth of the cuts (dimension 558 and 553) may be controlled to form a series of adjacent rings that are joined by beams 546 positioned on the opposite sides (e.g., 180 degrees apart) of the tube. The beams 546 carry forces across the cut area at that location along the longitudinal axis 548 of the tube. The beams 546 carry or transfer forces in roughly an axial direction from one ring to an adjacent ring on the opposite side of each beam 546. In one embodiment, adjacent pairs cuts 545, 554, and 550 are made in a pattern of alternating orientations along the length of tube 206. For example, the angle of the pair of cuts 554 with respect to the pairs cuts on either side may be 90 degrees. This is done, for example, by rotation of the tube 206 relative to the machine used for cutting. The tube 206 can be cut by two saws that approach the tube 206 from directly opposite sides or by any appropriate method. For example, cutting may be done by laser, by electric discharge or by punching. Each succeeding pair of cuts may be shifted angularly from the prior set of cuts. For example, the process may begin randomly by making cuts on the right and left sides; then, the next cuts will be made on the top and bottom sides; then, once again, from the right and left sides. Each successive pair of cuts may be angularly displaced anywhere from 0° to 90°, so that successive beams are located at the same location (0°), at perpendicular angles to one another (90°), or any amount of displacement from 0° and 90°. For example, the cuts may alternate repeatedly from one to the next in amounts such as 0 degrees, 22.5 degrees, 45 degrees, 67.5 degrees, 90 degrees, or any multiple thereof. The amount of rotation is selected with each successive cut to give a pattern calculated to facilitate torque transmission while also facilitating flexing of the tube. The result is a tube having a number of axial rings 546 that are joined by transverse beams 552. The rings 552 are generally defined by the curved portion of the tube wall between the adjacent cuts. As will be appreciated, these rings carry forces from a particular set of axial beams to the two adjacent sets of axial beams.

In order to optimize the tube for maximum torque transmission, the goal is to match the strain and the dimensions of the rings and beams along the length of the tube 206. This is to avoid a weak point in the material that may fail by deformation when torquing forces are applied. The torque tube prevents the shaft from deforming significantly so that steering becomes possible through rotation of the shaft. A single control cable or a pair of control cables used in combination with the torque tube may be sufficient. The matching of forces to a suitably rigid structure of transverse rings and axial beams can be done in tubes of constant wall thickness by variation of several parameters, namely the spacing dimension 555 between cuts, the cut width dimension 556 of each cut, and cut depth dimension 558. In hollow tubes of varying wall thickness, the wall thickness should to be taken into consideration, and may also be varied. Wider spacing of cuts creates wider rings; shallower cuts create wider axial beams. Likewise, more closely spaced cuts create narrower rings and deeper cuts create more narrow axial beams.

In manufacturing the hollow torque tube, a saw blade of a specified width can be used. Accordingly, the width of all cuts is held to this value. A diamond-silicon wafer cutting saw blade (as is used in the microprocessor and memory chip manufacturing sector) about one-thousandth of an inch wide is used to make the cuts. While it is possible to make wider cuts by making a first cut then moving the tube relative to the blade by a distance up to a width of the blade and repeating as necessary for wider cuts, speed of fabrication is higher if a single cut is used.

In arriving at the proper depth and spacing, dimensions need to be selected. The locations of the axial beams 546 will usually be determined by the relative angular displacement of the adjacent sets of opposed cuts and, hence, the width and the length of the rings 552 will be known. The width of the axial beams to be created depends on the depth of the cut. The length of each axial beam is the same and equal to the constant cut width of the saw blade.

Other factors are taken into consideration. For example, there is a practical limit on the size of rings and transverse beams with respect to the tube material and geometry. Too large and the desired advantages are lost; too small and imperfections in materials and variations within the tolerances in machining can compromise performance. This may be governed by the thickness of the tube wall, the size of the saw blade, accuracy of the machining apparatus, etc. Generally speaking, axial or transverse beams having dimensions on a par with or smaller than the width of the cutting blade used to micromachine them are to be avoided.

The design process then, in summary, is to space the cuts along the axis 548 of the tube 206 so as to provide flexing as desired. The cuts will be closer together to give less resistance to bending, and spaced farther apart to provide more resistance to bending. The stiffness can be controlled by varying the spacing of the cuts, the other parameters being selected, as appropriate. The bending stiffness of the tube 206 can vary along the longitudinal axis, for example, being made to gradually become less stiff toward the distal end by gradually decreasing the spacing between cuts.

While illustrative embodiments have been illustrated and described, it will be appreciated that various changes can be made therein without departing from the scope of the invention as defined by the following claims and equivalents thereof.

What is claimed is:

1. A medical device, comprising:
a flexible shaft having a proximal section and a distal section with a distal tip;
a control cable having one end secured at or adjacent the distal tip of the shaft that when activated bends the distal tip of the distal section in a desired direction; and
a flexible torque tube comprising at least one lumen along the length of the shaft from the proximal section to the distal section, wherein the tube is torsionally rigid to transfer torque forces from the proximal section to the distal section to change the direction of the distal tip when the distal tip is in a flexed configuration, without significant deflection of the torque tube circumferentially.

2. The medical device of claim 1, further comprising a handle having a distal end connected to the proximal section of the shaft, wherein the torque tube extends from the distal end of the handle to the distal tip of the flexible shaft.

3. The medical device of claim 1, wherein the torque tube comprises a series of cuts separated by axial beams along the length of the tube.

4. The medical device of claim 3, wherein the cuts alternate in angular displacement along the length of the torque tube.

5. The medical device of claim 1, wherein the torque tube is made from a metal or polymer.

6. The medical device of claim 1, comprising a control cable extending lengthwise from the proximal section to the distal section of the shaft to flex the distal tip.

7. The medical device of claim 6, comprising a steering dial that is connected to the control cable.

8. The medical device of claim 1, comprising a multi-lumen tube comprising two lumens and a control cable within each of the two lumens.

9. The medical device of claim 1, comprising a sheath on the exterior of the shaft.

10. The medical device of claim 9, further comprising a braid mesh material adjacent and interior to the sheath, wherein the torque tube is adjacent and interior of the braid mesh material.

11. The medical device of claim 10, further comprising a multi-lumen tube interior to the torque tube.

12. The medical device of claim 9, wherein the sheath and multi-lumen tube are made from an elastomer.

13. The medical device of claim 1, wherein the torque tube comprises a series of rings connected by beams.

14. The medical device of claim 13, wherein the beams are axial beams.

15. The medical device of claim 13, wherein the beams are at an angle to a longitudinal axis of the torque tube.

16. The medical device of claim 1, wherein the torque tube comprises sets of axial beams that alternate orientation at each successive cut in the tube, the orientation being any one of 0 degrees, 22.5 degrees, 45 degrees, 67.5 degrees, 90 degrees from the adjacent cut, or any multiple thereof.

17. The medical device of claim 1, wherein the torque tube comprises cuts of constant width.

18. The medical device of claim 1, wherein the torque tube comprises sets of axial beams along the length of the tube, wherein each set of axial beams is oriented from 0 degrees to 90 degrees from the adjacent set.

19. A medical device, comprising:
a flexible shaft having a proximal section and a distal section with a distal tip;
a control cable that steers the distal section in a direction; and
a flexible torque tube within the shaft that transfers a torque force from the proximal section to the distal section without significant deflection of the tube circumferentially so as to rotate the distal section.

20. The medical device of claim 19, wherein the torque tube comprises a series of cuts separated by beams along the length of the tube.

21. The medical device of claim 20, wherein the cuts alternate in angular displacement along the length of the torque tube.

22. The medical device of claim 19, wherein the torque tube is made from a metal or polymer.

23. The medical device of claim 19, comprising a pair of control cables placed approximately opposite to one another that extend lengthwise from the proximal section to the distal section of the shaft to flex the distal tip.

24. The medical device of claim 23, comprising a steering dial that is connected to the pair of control cables.

25. The medical device of claim 19, comprising a multi-lumen tube having two lumens and a control cable within each of the two lumens.

26. The medical device of claim 19, comprising a sheath on the exterior of the shaft.

27. The medical device of claim 26, further comprising a braid mesh material adjacent and interior to the sheath, wherein the torque tube is adjacent and interior of the braid mesh material.

28. The medical device of claim 26, further comprising a multi-lumen tube interior to the torque tube.

29. The medical device of claim 28, wherein the sheath and multi-lumen tube comprise an elastomer.

30. The medical device of claim 19, wherein the torque tube comprises a series of transverse beams connected by the axial beams.

31. The medical device of claim 19, wherein the torque tube comprises sets of axial beams that alternate orientation at each successive cut in the tube, the orientation being any one of 0 degrees, 22.5 degrees, 45 degrees, 67.5 degrees, 90 degrees from the adjacent cut, or any multiple thereof.

32. The medical device of claim 19, wherein the torque tube comprises cuts of constant width.

33. The medical device of claim 19, wherein the torque tube comprises sets of axial beams along the length of the tube, wherein each set of axial beams is oriented from 0 degrees to 90 degrees from the adjacent set.

34. A method for making a shaft of a medical device, comprising:
machining a tube to provide cuts in an alternating sequence along the length of the tube; and
assembling the cut tube within an elongated shaft, wherein the tube extends the majority of the length of the shaft, and other medical device components wherein at least one other component is a control cable to provide a steerable and torqueable medical device.

35. The method of claim 33, wherein the tube comprises transverse beams connected by axial beams.

36. The method of claim 33, wherein medical device components include at least one of a sheath, braid mesh, and multi-lumen tube.

37. The method of claim 33, wherein the tube is cut by a saw blade.

38. The method of claim 33, wherein any one of the cut width, cut depth, cut orientation, and cut spacing is adjustable to control the degree of flexing.
